(19) United States
(12) Patent Application Publication
(10) Pub. No.: US 2021/0155896 A1
(43) Pub. Date: May 27, 2021

(54) SINGLE BRAIN CELL-DERIVED ORGANOIDS

(71) Applicant: Rutgers, The State University of New Jersey, New Brunswick, NJ (US)

(72) Inventor: Hatem Sabaawy, Neshanic Station, NJ (US)

(21) Appl. No.: 16/625,969

(22) PCT Filed: Jun. 28, 2018
(86) PCT No.: PCT/US18/39994

§ 371 Date: Dec. 23, 2019

Related U.S. Application Data
(60) Provisional application No. 62/526,068, filed on Jun. 28, 2017.

(51) Int. Cl. C12N 5/00; A01K 67/027

(52) U.S. Cl. CPC C12N 5/0062; A01K 67/0278; C12N 2501/11; A01K 2227/105; C12N 2501/999; A01K 2267/0331; C12N 2501/115

(57) ABSTRACT

The present invention relates to organoids derived from a single brain cell, such as, for example, a Glioblastoma (GBM) cell, and methods and compositions relating to the production and use thereof, including cell culture medium for producing organoids and methods of personalized treatment for GBM cancer and other brain disorders. The invention further provides a humanized mouse including a GBM organoid derived from a patient's GBM cell.

### CROSS-REFERENCE TO RELATED APPLICATION

This application claims the benefit of U.S. Provisional Application No. 62/526,068 filed Jun. 28, 2017, the disclosure of which is incorporated herein by reference in its entirety.

### BACKGROUND OF THE INVENTION

The inability to propagate primary tissues represents a major hurdle to understanding the mechanisms of regeneration and the balance of differentiated cells versus stem cells in adult organisms. A need exists to better understand primary human pathological disorders such as injury repair and tumor development. For cancer studies, current cancer models do not adequately represent the molecular and cellular diversity of human cancers. Existing human cancer cell lines lack defined and detailed information regarding the clinical presentation of the cancer and have inherent limitations for deciphering the mechanisms of therapy resistance. For injury repair, there is a lack of understanding of the mechanisms of regeneration and shortage of tissue and organs for transplantation. Therefore, novel methods to maintain primary tissues for cancer, new drug discovery approaches to treat cancer and regenerative medicine indications are needed.

Maintaining the balance between normal differentiated cells and progenitor or stem cells is complex. Adult stem cells provide regeneration of different tissues, organs, or neoplastic growth through responding to cues regulating the balance between cell proliferation, cell differentiation, and cell survival, with the later including balanced control of cell apoptosis, necrosis, senescence and autophagy. Epigenetic changes, which are independent of the genetic instructions but heritable at each cell division, can be the driving force towards initiation or progression of diseases. Tissue stem cells are heterogeneous in their ability to proliferate, self-renew, and differentiate and they can reversibly switch between different subtypes under stress conditions. Tissue stem cells house multiple subtypes with propensities towards multi-lineage differentiation. Hematopoietic stem cells (HSCs), for example, can reversibly acquire three proliferative states: a dormant state in which the cells are in the quiescent stage of the cell cycle, a homeostatic state in which the cells are occasionally cycling to maintain tissue differentiation, and an activated state in which the cells are cycling continuously. The growth and regeneration of many adult stem cell pools are tightly controlled by these genetic and/or epigenetic responses to regulatory signals from growth factors and cytokines secreted through niche interactions and stromal feedback signals.

Glioblastoma (GBM) (also known as glioblastoma multiforme) is the most frequent and lethal brain cancer. About 22,000 Americans are diagnosed with GBM annually. Lack of early detection methods and rapid growth kinetics, with most patients dying within 2 years, make this cancer especially deadly. The frequent GBM recurrence is derived in large part by the marked radio- and chemo-resistance. Therapeutic resistance is likely due to multiple factors within the GBM tumor, but several studies suggested that subpopulations of cancer cells in GBM (i.e. Brain cancer stem-like cells or BCSCs) are highly resistant to radiation and chemotherapies. Improved GBM therapies to target BCSCs that are examined in patient-derived models are greatly needed.

### SUMMARY OF THE INVENTION

In one embodiment, the present invention provides a method of making an organoid from a mammalian brain tissue in vitro comprising: isolating cells from a mammalian GBM tissue to provide isolated cells; culturing the isolated cells in a differentiation medium for a time sufficient to enrich for stem cells and induce differentiation; and amplifying the cells by culturing in an extracellular matrix in an organoid medium for a time sufficient to produce organoids that exhibit endogenous three-dimensional organ architecture.

In another embodiment, the invention provides an in vitro GBM organoid comprising brain cancer stem-like cells (BCSCs) and their differentiated progeny, the organoid exhibiting endogenous three-dimensional organ architecture.

In one embodiment, the in vitro GBM organoid is derived from a single cell of a brain tissue and exhibits endogenous three-dimensional organ architecture.

In another embodiment, the invention provides an in vitro brain organoid derived from primary brain normal tissue, wherein the organoid comprises normal brain neural cells and exhibits endogenous three-dimensional organ architecture. Methods of distinguishing brain tumor tissue from normal brain tissue at the tumor margin are known in the art using spectral and fluorescence imaging and disclosed for example Kaur et al. (2017) Scientific Reports 6:26538 and Hollon et al. (2016) Neurosurg Focus 40:e9. In accordance with one aspect of the present invention, brain tumor cells and normal brain cells can be derived from resected glioblastoma tissues from patients. Methods of distinguishing GBM tumor tissue from normal brain tissue using stimulated raman spectroscopy and fluorescent multimodal imaging are also known in the art and disclosed for example by Zanello et al. (2017) Scientific Reports 7:41274.

In another embodiment, the invention provides an in vitro GBM organoid derived from primary GBM cancer tissue, wherein the organoid comprises BCSCs and exhibits endogenous three-dimensional organ architecture.

In another embodiment, the invention provides a cell culture medium supplemented with B27 supplement, basic fibroblast growth factor (bFGF), and epidermal growth factor (EGF). B27 supplement is commercially available from Gibco™ as B-27™ Supplement (50×).

In another embodiment, the invention provides a cell culture medium supplemented with B27 supplement, bFGF, EGF, and hydrocortisone.

In another embodiment, the invention provides a cell culture medium additionally supplemented with Penicillin and Streptomycin.

In another embodiment, the present invention provides a kit including a cell culture medium supplemented with B27 supplement, bFGF, and EGF, and a cell culture medium supplemented with B27 supplement, bFGF, EGF, and hydrocortisone.

In another embodiment, the invention provides a method for identifying agents having anticancer activity against GBM cells including selecting at least one test agent, contacting a plurality of patient-specific GBM organoids derived from the patient's GBM cell with the test agent, determining the number of GBM organoids in the presence of the test agent and the absence of the test agent, and identifying an agent having anticancer activity if the number or the growth of the organoid cells is less in the presence of the agent than in the absence of the agent. In another embodiment, the method provides a step of treating the patient with the agent identified as having anticancer activity against the patient-specific organoids but not against normal organoids. A method for identifying agents having anticancer activity against GBM cells can further include providing a mouse engrafted with GBM cells from the patient and containing a tumor formed from the GBM cells; administering the identified agent having anticancer activity to the mouse; and determining if the tumor size is reduced in the presence of the identified agent. In another embodiment, a method for identifying agents having anticancer activity against GBM cells can further include providing a humanized mouse engrafted with components of a patient's immune system and GBM cells from the patient and containing a tumor formed from the GBM cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered. This and other embodiments can further include providing a humanized mouse engrafted with GBM cells from the patient and containing a tumor formed from the GBM cells; administering a control agent to the humanized mouse engrafted with GBM cells from the patient; and comparing the size of the tumor in the humanized mouse engrafted with GBM cells from the patient to the size of the tumor in the mouse in which the identified agent was administered; and determining if the size of the tumor in the mouse in which the identified agent was administered is reduced relative to the size of the tumor in the humanized mouse engrafted with GBM cells from the patient.

In another embodiment, the method provides a step of treating the patient with the agent identified as having anticancer activity against the patient-specific organoids but not against normal organoids.

In another embodiment, the present invention provides normal patient-specific brain organoids, and methods of using such organoids for personalized therapies for neural tissue regeneration and developing new therapies for neurological disorders.

In another embodiment, the present invention provides immune humanized mice with implanted patient-specific GBM organoids, and methods of using such mice to identify personalized therapies for GBM.

In the methods described herein, the organoids exhibit endogenous three-dimensional organ architecture.

### BRIEF DESCRIPTION OF THE FIGURES

FIGS. 1A-1D are schematic illustrations and histological images showing histologic and immunophenotypic parity between original patient GBM tissue and organoids and cells used to generate orthotopic xenografts in the mouse brain. FIG. 1A: Strategy to generate spheres and patient derived orthotopic xenografts (PDOXs) from primary GBM tissue. The diagram displays the process of microinjecting sphere cells into the cerebrum region of the mouse brain. The location of burr drilling hole in NSG mice skull is demonstrated for microinjections using stereotactic infusion pump resulting in effective (90% take) generation of orthotopic GBM PDOXs. FIG. 1B: Histological H&E analysis of original patient derived GBM tissue (patient #46) and four different PDOX lines generated from the same patient-derived spheres. Note that the cell density is different in these sections as it depends on the number of cells engrafted into the PDOXs. The lower panels are 1000× higher magnification of the outlined areas in the top panels. FIG. 1C: Representative sections for comparison of the expression of stem cell proteins (BMI1, NESTIN and SOX9) and the proliferation marker Ki67 in the original patient GBM tissue and the corresponding PDOX.

FIG. 1D: Representative sections for comparison of the clustered expression of the GBM stem cell marker (CD15) in vascular niches and with the hypoxia protein Carbonic Anhydrase IX (CA9) near blood vessels expressing CD31, both in the original patient GBM tissue and the corresponding PDOX. Scale bars are 500 μM in the upper panel of B and 20 μM in the lower panel of FIG. 1B, FIG. 1C and FIG. 1D.

### DETAILED DESCRIPTION OF THE INVENTION

In certain embodiments, the present invention provides GBM organoids derived in vitro from cancerous tissues and brain organoids derived in vitro from normal tissues, and methods of making and using such organoids, as well as cell culture media and kits. As disclosed in one embodiment herein, certain growth factors in an in vitro environment containing extracellular matrix molecules in a 3-dimensional culture device may be used to make the organoids.

An organoid is a miniature form of a tissue that is generated in vitro and exhibits endogenous three-dimensional organ architecture. See, e.g., Cantrell and Kuo (2015) Genome Medicine 7:32-34. The organoids of the present invention can be used, for example, to: a) determine genomic targets within tumors and prediction of response to therapies in preclinical and clinical trials; b) detect the activity of an anti-cancer agent by examining the number of surviving organoids after treatment; c) detect the activity of a proliferative agent by determining the number of proliferating cells within each organoid and determining gene expression profiling of relevant pathways; d) examine the specificity of agents targeting different cell types within organoids; e) determine the effects of chemotherapy and radiation; f) create mouse models by implantation of the organoid in vivo; g) create preclinical models for examining therapy responses and drug discovery both in vitro and in vivo; and h) determine clonally-targeting anti-cancer therapies.

Accordingly, in one embodiment, the invention provides a method of making an organoid from a mammalian brain tissue in vitro including: isolating cells from a mammalian brain tissue to provide isolated cells; culturing the isolated cells in a differentiation medium for a time sufficient to enrich for stem cells and induce differentiation; and amplifying one or more of the cells by culturing in an extracellular matrix in an organoid medium for a time sufficient to produce organoids. One of ordinary skill in the art can determine a time sufficient to induce differentiation by examining morphological changes associated with differentiation. In one preferred embodiment, the time sufficient to enrich for stem cells and induce differentiation is from about 7 to about 180 days. In another preferred embodiment, the time sufficient to induce differentiation is about 7 days. One of ordinary skill in the art can determine a time sufficient to induce organoid formation by examining morphological changes associated with organoid formation. In one preferred embodiment, the time sufficient to induce organoid formation is from about 5 to about 28 days. In another preferred embodiment, the time sufficient to induce organoid formation is about 14 days. In one embodiment, the isolated cells are GBM cells. In one embodiment, a single GBM cell is amplified.

In one preferred embodiment, the differentiation medium comprises Eagle's minimum essential medium (EMEM) (ThermoFisher Scientific), B27 supplement, bFGF, and EGF. EMEM and B27 supplement are typically used at 1×. However, the concentration of B27 supplement present in the differentiation medium may range from about 0.5× to about 5×. The concentration of bFGF present in the differentiation medium may range from about 0.1-100 mg/mL (e.g., 1 mg/mL, 5 mg/mL, 10 mg/mL, 15 mg/mL, 20 mg/mL, etc). The concentration of EGF present in the differentiation medium may range from about 0.1-100 mg/mL (e.g., 1 mg/mL, 5 mg/mL, 10 mg/mL, 15 mg/mL, 20 mg/mL, 25 mg/mL, etc). In a further embodiment, the differentiation medium comprises one or both of Penicillin (500-5000 Units/mL) and Streptomycin (50-500 μg/mL). In a most preferred embodiment, the differentiation medium comprises the following concentrations: Dulbecco's Modified Eagle Medium: Nutrient Mixture F-12 (DMEM/F12) (ThermoFisher Scientific) (about 1×); B27 supplement (about 1×); bFGF (about 10 mg/mL); EGF (about 20 mg/mL); Penicillin (about 1000 Units/mL); and Streptomycin (about 100 μg/mL). The differentiation medium may further comprise or be substituted with other supplements, growth factors, antibiotics, vitamins metabolites, and hormones, synthetic or natural with similar properties as known in the art.

In one preferred embodiment, the organoid 3D culture medium includes EMEM, B27 supplement, bFGF, EGF, and hydrocortisone. Eagle's minimum essential medium (EMEM) (ThermoFisher Scientific) and B27 supplement are typically used at 1X. However, the concentration of B27 supplement present in the organoid medium may range from about 0.5× to about 5×. The concentration of bFGF present in the organoid medium may range from about 0.1-100 mg/mL (e.g., 1 mg/mL, 5 mg/mL, 10 mg/mL, 15 mg/mL, 20 mg/mL, etc). The concentration of EGF present in the organoid medium may range from about 0.1-100 mg/mL (e.g., 1 mg/mL, 5 mg/mL, 10 mg/mL, 15 mg/mL, 20 mg/mL, 25 mg/mL, etc). The concentration of hydrocortisone present in the organoid medium may range from about 0.1-10 mM (e.g., 0.1 mM, 0.5 mM, 0.75 mM, 1 mM, 1.5 mM, 2 mM, 5 mM, etc). In a further embodiment, the organoid medium further includes Penicillin (about 500-5000 Units/mL), and Streptomycin (about 50-500 μg/mL). In a most preferred embodiment, the organoid medium includes the following concentrations: EMEM (ThermoFisher Scientific) (about 1×); B27 supplement (about 1×); bFGF (about 10 mg/mL); EGF (about 20 mg/mL); about 1 mM hydrocortisone; Penicillin (about 1000 Units/mL); and Streptomycin (about 100 μg/mL). The organoid medium may further include or be substituted with other supplements, growth factors, antibiotics, vitamins metabolites, and hormones, synthetic or natural with similar properties as known in the art.

In some embodiments, the cells are cultured in organoid medium using a bioreactor (e.g., a spinning bioreactor) after the organoids are formed in a multiwell plate(s). An experiment was performed to increase the size of the formed organoids. After 4-6 days of 3D culture in organoid chamber droplets in a multiwell plate, the droplets were transferred to a spinning bioreactor. The organoids cultured in the spinning bioreactor became 3-10 fold larger in size than those cultured in multiwell plates at 30-60 days after culture. Thus, a spinning bioreactor may be used in some embodiments.

In a typical culture medium as described herein (differentiation medium, organoid medium), B27 is used, as experiments performed by expanding the cells from two GBM tumors in B27 (minus vitamin A) for 4 days then replacing the media with B27 (plus vitamin A), found the latter method (B27 plus vitamin A) to produce as many and larger organoids. However, in some alternative embodiments, B27 minus vitamin A can be used in a culture medium as described herein.

In certain embodiments, the cells are from human brain tissue, and human primary GBM tissue. In certain embodiments, cells that may be used to make an organoid are human BCSCs. Such cells are known in the art and may be identified and isolated using markers, for example, CD133, CD15, CD24, CD151, SOX2, OLIG2, ZEB1, NESTIN, BMI1, PTEN, and GFAP. Such cells may be identified and isolated by methods of cell sorting that are known in the art. For example, in one embodiment, the cells may be isolated by cell sorting for CD15 or RNA sorting using methods known in the art, such as molecular beacons and the SmartFlare™ probe protocol (EMD Millipore).

In one preferred embodiment, the cells are obtained from surgically excised tissues by subjecting the tissues to mechanical dissociation and filtration.

In one preferred embodiment, the cells are cultured in poly-ornithine coated plates for the time sufficient to enrich for stem cells and induce differentiation.

In certain embodiments the method is performed with a commercially available extracellular matrix such as Matrigel™. Other natural or synthetic extracellular matrices are known in the art for culturing cells. In general, an extracellular matrix comprises laminin, entactin, and collagen. In a preferred embodiment the method is performed using a 3-dimensional culture device (chamber) that mimics an in vivo environment for the culturing of the cells, where preferably the extracellular matrix is formed inside a plate that is capable of inducing the proliferation of stem cells under hypoxic conditions. Such 3-dimensional devices are known in the art. An example of such a device is disclosed by Bansal, N., et al. (2014) Prostate 74, 187-200, the disclosure of which is incorporated herein by reference in its entirety. It has been discovered in accordance with the present invention that the use of a 3-dimensional culture device in a method of making organoids has surprising advantages over other formats, as shown in Table 1.

TABLE 1
Advantages and disadvantages of tested formats

| | Consistency | | |
|---|---|---|---|
| Format | of Organoids | Reproducibility | Efficiency |
| In Matrigel ™ | +++ | +++ | ++++ |
| On Matrigel ™ | + | −−− | ++ |
| Hanging Drop plates | −−− | −−− | −−− |
| Non adherent plate | + | −−− | + |

In another aspect, the invention provides a brain tissue organoid. The brain tissue organoids of the present invention resemble the structures of the primary tissue. Upon histological and immunofluorescence analyses, one of skill in the art can determine that the organoids recreate the human neural tissues. Brain tissue origin of organoids can be confirmed by detecting the expression of NESTIN, TUBULIN, GAL-C and GFAP.

In another aspect, the invention provides a brain cancer organoid derived in vitro from primary GBM tissue. Tumor heterogeneity can be efficiently modeled using the methods described to make an organoid, by mapping the diagnostic dominant clone and tumor subclones from each patient biopsy sample, generating organoids derived from each clone and defining the genetic signature of each clone. A GBM organoid derived from primary GBM tissue will generally maintain expression of GBM lineage-specific markers, be capable of interconnecting (mimicking brain cells) and differentiating into cells with multiple cell phenotypes, and have BCSC-like features. A GBM organoid as described herein can be serially propagated, cryofrozen and regenerated and established as a model for cancer drug discovery and precision therapy.

In another aspect, the invention provides a GBM organoid derived in vitro from surgically excised tissues of tumors identified to express histopathological tissue specific and tumorigenic markers. Single cells from these tissues may be isolated with non-contact laser capture microdissection, cell sorting or by RNA sorting, for example using SmartFlare™ probes to generate single cell organoids with known expression features.

The organoids described herein exhibit endogenous three-dimensional organ architecture.

In another embodiment, the invention provides a method for identifying agents having anticancer activity against GBM cells from a patient(s) including selecting at least one test agent, contacting a plurality of patient-specific GBM organoids derived from the patient's GBM cell with the test agent, determining the number of GBM organoids in the presence of the test agent and the absence of the test agent, and identifying an agent having anticancer activity if the number or growth of the organoids is less in the presence of the agent than in the absence of the agent. In another embodiment, the method provides a step of treating the patient with the agent identified as having anticancer activity against the patient-specific organoids. A method for identifying agents having anticancer activity can further include providing a mouse engrafted with GBM cells from the patient and containing a tumor formed from the GBM cells; administering the identified agent having anticancer activity to the mouse; and determining if the tumor size is reduced in the presence of the identified agent.

A method for identifying agents having anticancer activity can further include providing a humanized mouse engrafted with components of a patient's immune system and GBM cells from the patient and containing a tumor formed from the GBM cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered. In this embodiment, the humanized mice with the patient's immune system can be used to compare the effects of the identified agent (e.g., candidate therapeutic) on tumors in the presence or absence of immune cells to examine a potential role for combination with immunotherapy. These methods can further include providing a mouse (an immune-deficient control mouse) engrafted with GBM cells from the patient and containing a tumor formed from the GBM cells; administering a control agent to the mouse engrafted with GBM cells from the patient; and comparing the size of the tumor in the mouse engrafted with GBM cells from the patient to the size of the tumor in the mouse in which the identified agent was administered; and determining if the size of the tumor in the mouse in which the identified agent was administered is reduced relative to the size of the tumor in the mouse engrafted with GBM cells from the patient in which a control agent was administered. In this method, if the size of the tumor in the mouse in which the identified agent was administered is reduced relative to the size of the tumor in the mouse in which a control agent was administered, the identified agent can be confirmed as a successful treatment for cancer in the patient.

In another embodiment, the invention provides a method of selecting a personalized treatment for GBM in a subject including: selecting at least one form of treatment, contacting a plurality of GBM organoids with the form of treatment, wherein the organoids are derived from GBM cells from the subject, determining the number of GBM organoids in the presence of the treatment and the absence of the treatment, and selecting the treatment if the number or growth of the GBM organoids is less in the presence of the treatment than in the absence of the treatment. Various types of therapy can then be examined using the organoids to determine therapy resistance before initiation, to tailor the therapy for each individual patient based on oncogenic driver expression in the organoids, as well as further study induced clonal selection processes that are the frequent causes of relapse. Various forms, combinations, and types of treatment are known in the art, such as radiation, hormone, chemotherapy, biologic, and bisphosphonate therapy. The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. Terms such as “treating” or “treatment” or “to treat” or “alleviating” or “to alleviate” refer to therapeutic measures that cure, slow down, lessen symptoms of, and/or halt progression of a diagnosed pathologic condition.

The foregoing methods may be facilitated by comparing therapeutic effects in organoids derived from cancer cells and normal cells from the same patient. For example, paraffin-embedded tissue, normal organoids and cancer organoids derived from cells of the same patient can be assessed to determine genetic and epigenetic mutations and gene expression profiles that are cancer-specific, thereby allowing the determination of gene-drug associations and optimization of treatment. Such comparisons also allow one to predict a therapeutic response and to personalize treatment in a specific patient.

In another aspect of this method, clonally targeted therapies can be determined by testing the effect of a therapeutic agent on multiple organoids derived from subsequently determined dominant clones of GBM cells identified in the original or recurrent tumor tissue from a patient, and comparing to the effect of the therapeutic agent on organoids derived from normal cells of the same patient to identify the features of resistant cells and determine tumor evolution.

In another aspect, the invention provides a cell culture (e.g., organoid) medium supplemented with B27 Supplement. In another aspect, the invention provides a cell culture (e.g., organoid) medium supplemented with B27 Supplement, bFGF, EGF and hydrocortisone. In another embodiment, the invention provides a cell culture (e.g., organoid) medium supplemented with B27 Supplement, bFGF, EGF, hydrocortisone, Penicillin and Streptomycin. In a preferred embodiment, the medium is a commercially available cell growth medium such as EMEM (ThermoFisher Scientific).

In another aspect, the invention provides kits to make an organoid from a single cell. In an embodiment, a kit contains containers for a differentiation medium and an organoid medium as previously described. The containers may also contain the necessary supplements (growth factors, antibiotics, hormones, vitamins, amino acids, and combinations thereof) for a differentiation medium and an organoid medium. The kit may further include the necessary components for a 3-dimensional culture device, for example, plates, and/or materials for an extracellular matrix, e.g. Matrigel™. The kit may further contain a set of instructions to perform the methods of making an organoid from a single cell as previously described.

In another embodiment, the present invention provides a mouse with an implanted patient-specific GBM organoid. In one embodiment, the mouse is a humanized mouse. In another embodiment, the mouse is a human immune system (HIS)-reconstituted mouse. In another embodiment, the mouse is non-obese diabetic (NOD)-Rag (−)-γ chain (−) (NRG) mouse. In another embodiment, the mouse is an NSG immune-deficient PDX mouse.

Methods of making HIS-reconstituted mice are known in the art and disclosed for example by Drake et al. (2012) Cell Mol Immunol 9:215-24 and Harris et al. (2013) Clinical and Experimental Immunology 174:402-413. In accordance with one aspect of the present invention, human stem cells from patient, for example from a diagnostic bone marrow or blood sample or HLA-matched, are transplanted into neonatal NRG mice to engraft components of the patient's immune system. Methods of making NSG immune-deficient PDX mice are also known in the art and disclosed for example by Jarzabec et al. (2013) Mol Imaging 12: 161-172. The mice are later subjected to grafting with GBM organoids derived from GBM cells of the same patient orthotopically in the mouse brain. The mice are useful for identifying new treatments, assessing responses to therapy, and evaluating combination therapies.

The following non-limiting examples serve to further illustrate the invention.

### Example 1

To generate GBM organoids, a two-step methodology was used, including a first phase to enrich for BCSCs, conducted in a two-dimensional (2D) setting (stage I), followed by a second phase of organoid 3D growth obtained in pure matrigel chambers (stage II). Niche growth factor supplementation specific to brain-derived tissue was used. Table 2 below includes the media and culture conditions in a typical embodiment of producing GBM tissue organoids.

TABLE 2
GBM Organoid Media

| | | | | 3D Culture | |
|---|---|---|---|---|---|
| Primary | Collection | Process | 2D Culture | (Phase II in | Days to |
| Tissue | Media | Time | (phase I) | Matrigel) | organoids |
| GBM | DMEM/F12 | Mechanical | EMEM | EMEM | Phase I: 7 |
| | medium + | dissociation, | medium + | medium + B27 | Phase II: 14 |
| | B27 + bFGF | culture for a | B27 (1X, | (1X, Gibco) + | |
| | (10 mg/mL) + | day, then | Gibco) + | bFGF | |
| | EGF | Accutase, | bFGF | (10 mg/mL) + | |
| | (20 mg/mL) + | passed in a | (10 mg/mL) + | EGF | |
| | Penicillin | needle to | EGF | (20 mg/mL) + | |
| | (3,000 | single-cell | (20 mg/mL) + | Hydrocortisone | |
| | Units/mL) + | suspension. | Penicillin | (1 mM) + | |
| | Streptomycin | | (1,000 | Penicillin | |
| | (300 μg/mL) | | Units/mL) + | (1,000 | |
| | | | Streptomycin | Units/mL) + | |
| | | | (100 μg/mL) | Streptomycin | |
| | | | | (100 μg/mL) | |

### Example 2

De-identified primary human tumor samples were obtained from GBM patients undergoing craniotomy resection at Robert Wood Johnson University Hospital under an IRB approved protocol. Cells were obtained through mechanical dissociation of the tumor tissue using a blade and plated in different culture media. To enrich for GBM BCSCs, amenable for initiating cancer organoids, a two-step methodology was developed comprising a first phase of BCSC enrichment, conducted in a two-dimensional (2D) setting (stage I), followed by a second phase of organoid 3D growth obtained in pure matrigel chambers (stage II). Those tested for the first phase of 2D culture included Dulbecco's modified Eagle's medium (DMEM), Advanced DMEM (ADMEM), and DMEM/F12 medium. The following day, the culture was collected, incubated with accutase at 37° C. and passed through a needle to obtain a single cell suspension and re-plated in supplemented medium. Cells from primary tumors maintained as GBM organoids were grown in 6 well plates and were dissected into a single cell suspension using accutase (Gibco) and a syringe-needle. To establish 3D organoids, patient-derived cells from 2D cultures from four patients were utilized: patient GBM #46, patient GBM #50, patient GBM #70 and patient GBM #76. Upon completing the first phase, single cells were seeded at different clonal densities (100 or 500 cells/well) in each medium without serum in pure multilayer matrigel chambers. For the second phase 3D organoid culture, the presence and the number of organoids were then evaluated after 14 days. Three-dimensional culture methods recapitulate features of in vivo cell growth, allowing self-organization, differentiation, and mixed heterogeneity to exist within the culture environment were established in Matrigel chambers. DMEM/F12 medium in stage I in the presence of B-27 supplement, 20 ng/ml of both human recombinant EGF and human recombinant FGF, was found to be most supportive of generating GBM organoids after 14 days in stage II 3D culture. In the stage II setting, the organoids grew as compact structures, which could be expanded for multiple passages, and could be employed for in vitro assays. Cells were seeded at a clonogenic density (20 cells per well) into a 96 well plate; and number of secondary organoids formed per well was counted after 14 days. In matrigel, GBM cells form 3D structures. 3D cultured GBM organoids were capable of interconnecting (mimicking brain cells) and were also capable of differentiating into cells with multiple cell phenotypes. The clonogenic capabilities in 3D cultures differed between the 4 patients. To provide proof-of-concept that the GBM patient-derived organoids (PDOs) derived from each patient recreate the genomic profile of the parental cell of origin in the GBM specimen and have BCSC-like features to express different brain cell lineages, organoids grown in 3D matrix culture were stained with those in 2D differentiation culture or 3D organoid culture in immunofluorescence (IF) assays. Organoids grown in 3D matrix (matrigel) culture expressed the neural stem cell (NSC) marker NESTIN and Gal-C, and the neuronal marker tubulin and astrocyte marker GFAP. The 2D differentiated cells lacked NESTIN or Gal-C expression and showed TUBULIN expression, further validating the hypothesis that 3D organoid culture enriches for BCSC features. The presence of matrix is essential to maintain these expression features since 3D liquid cultures had different expression profiles.

To further demonstrate that these GBM organoid cultures were indeed enriched in clonogenic BCSC-like cells, the expression of multiple signaling effectors and BCSC markers between the original patient GBM tissue and 3D organoid cultures both in liquid and matrix phases were assessed. Among these, NESTIN, TUBULIN, GAL-C, BMI1, PTEN and GFAP are known to be vital for BCSC maintenance. It was observed that NESTIN, GAL-C, BMI1, PTEN and GFAP expression levels were all similar in GBM organoids vs. patient tissue, implying the presence of important BCSC fractions in the tumor organoid cultures. In conclusion, organoid forming conditions were developed for generating GBM organoids from multiple samples and it was further determined that they could be serially propagated, and regenerated into secondary organoids and contain BCSC features. This GBM organoid model is an outstanding resource to examine different therapies for GBM.

### Example 3

A BMI1 activity score was developed from primary GBM tissues for organoid drug sensitivity studies. Assessing the extent of BMI1 overexpression in GBM is vital for predicting sensitivity to BMI1- and BCSC-targeting drugs, but the best biomarker of BMI1 activity in FFPE tumor specimens and organoids is unclear. Organoid cultures were generated from GBM #46, #50, #70 and #76 specimens, which expressed NESTIN and retained the ability to differentiate into terminal lineages. Using patient-derived organoids, small molecules that target BMI1 in organoids and inhibit cellular self-renewal of BCSCs in 3D organoids were identified. The IC₅₀ values of these compounds were determined in U87 GBM cells and GBM organoids using MTT assays, and two small molecules were the most potent, but had no significant effects in normal astrocyte derived organoids. The selectivity and potency in U87 2D cell culture vs primary patient-derived GBM organoids was then assessed. Dose-response curves were generated in both organoids and monocultures and used to determine an organoid selectivity ratio (targeting BCSCs), defined as EC₅₀(U87)/EC₅₀(organoids), for each compound. Compounds that had an organoid selectivity ratio greater than controls (DMSO and Temozolomide) could be defined as agents with high selectivity for BCSCs (BMI1 inhibitors).

Self-renewal ability is a distinctive property of stem-like cells, for which BMI1 is a crucial regulator. To investigate the long-term impact of BMI1 inhibition, limiting dilution assays (LDA) in soft agar were performed to appraise differences in colony forming capacities. The collective results showed that, compared to controls, GBM colonies were nearly eliminated in the BMI1 inhibitor-treated cells (P<0.001).

To develop a combination therapy, patient-derived GBM organoids were treated with 3 Gy irradiation (IR) and BMI1-targeting therapy when lower BMI1 and p-AKT levels would render cells more vulnerable. The combination was more effective, and resulted in more senescent organoid cells, suggesting that the BMI1-targeting compounds might sensitize GBM cells to IR. Using patient-derived models would facilitate the translation of in vitro findings to in vivo discovery and maximize chances of future drug clinical success. While organoids preserve a tumor's 3D structure, orthotopic patient-derived xenografts replicate the in vivo tumor biology. An orthotopic GBM model was developed by transplanting organoids or spheres into NSG mouse brain and treatment of the animals with BMI1-targeting therapy resulted in a significant antitumor activity of BMI1 inhibitors against GBM #46 in mice. Moreover, cells from treated tumors had significantly less secondary organoid forming potential, demonstrating the depletion of BCSCs.

### Example 4

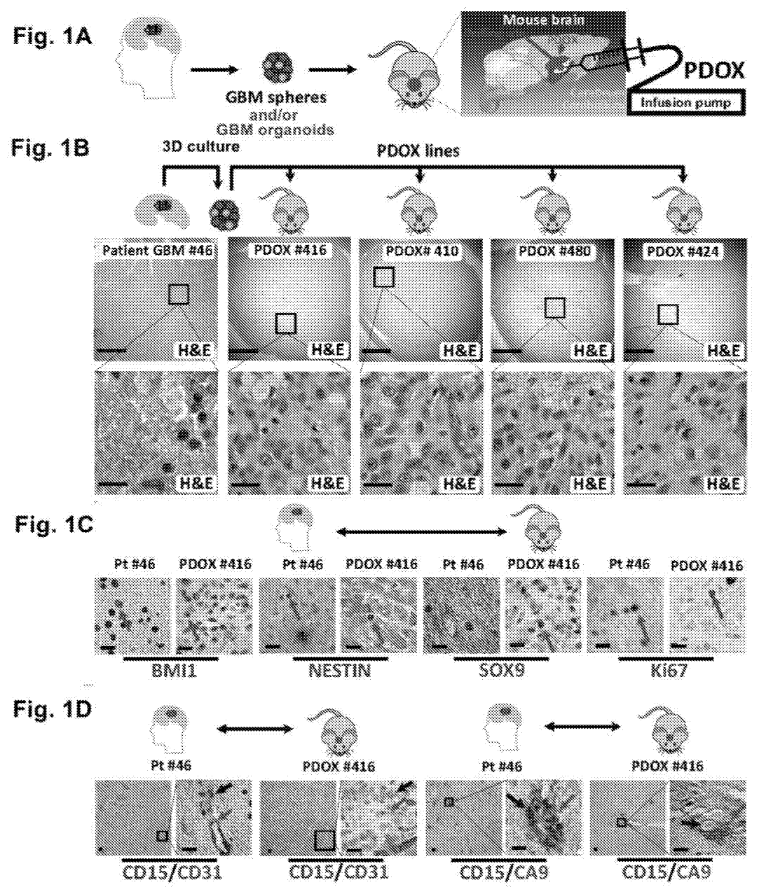

We generated data on the similarity and parity between the original patient tissue and the organoids formed in vitro in 3D culture by dissociating the primary tumor cells used to generate the organoids in vitro for intracranial injections to generate in vivo orthotopic xenografts in the mouse brain of immune deficient mice, to generate patient derived xenografts (PDXs) named patient derived orthotopic xenografts (PDOX) of the mouse brain. FIG. 1 shows that histological sections from the mouse brain in the PDOXs display cancer stem cell features like the original tissue from the patient surgical resection and express BCSC marker (CD15+) in cells surrounding the blood vessels (CD31+) and in necrotic areas (expressing CA9). Our data show comparable expression of BMI1, Nestin, Sox9, and Ki67 levels between sections from PDOXs and original patient tumor. These are characteristic features of GBM in patients; therefore, these data show that the organoid model described herein completely reproduces the key features of human tumors in vivo in mice.

Patient derived orthotopic xenografts (PDOXs) recapitulated key GBM morphological features (FIG. 1B), including necrosis and overall expression of the stem cell proteins BMI1, NESTIN and SOX9 and the proliferation marker Ki67 (FIG. 1C). Moreover, PDOXs demonstrated a high degree of hyperplastic blood vessels, a hallmark of GBM representing the original GBP patient tissues (FIG. 1D). Additionally, GBM cells expressing the BCSC protein CD15 were present in clusters of GBM niches in proximity of blood vessels, and cells expressing hypoxia protein Carbonic Anhydrase IX (CA9) were in close proximity to cells expressing CD15, within both original GBM patient tissue and corresponding PDOXs (FIG. 1D). Our studies demonstrate that PDOXs generated from patient-derived organoids make a reliable model for developing targeted and personalized therapies for GBM patients.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein by reference in their entireties.

We claim:

1. A method of making an organoid from a mammalian brain tissue in vitro comprising: isolating cells from mammalian brain tissue to provide isolated cells; culturing the isolated cells in a differentiation medium for a time sufficient to enrich for stem cells and induce differentiation; and amplifying one or more of the cells by culturing in an extracellular matrix in an organoid medium for a time sufficient to produce organoids that exhibit endogenous three-dimensional organ architecture.

2. The method of claim 1 wherein the differentiation medium comprises B27 Supplement, basic fibroblast growth factor (bFGF), and epidermal growth factor (EGF).

3. The method of claim 1 wherein the organoid medium comprises B27 Supplement, bFGF, EGF, and hydrocortisone.

4. The method of claim 3 wherein the organoid medium further comprises one or more of Penicillin and Streptomycin.

5. The method of claim 1 wherein the mammalian tissue is a human brain tissue.

6. The method of claim 5 wherein the human brain tissue is primary human normal brain tissue, or human Glioblastoma (GBM) tissue.

7. The method of claim 6 wherein the human GBM tissue is primary GBM cancer tissue.

8. The method of claim 1 wherein the organoids comprise brain cancer stem-like cells (BCSCs).

9. The method of claim 1 wherein the time sufficient to produce organoids is about fourteen days.

10. The method of claim 3 wherein the B27 Supplement is present at a concentration of about 0.5×-5×, the bFGF is present at a concentration of about 1-50 mg/mL, the EGF is present at a concentration of about 1-50 mg/mL, and the hydrocortisone is present at a concentration of about 0.1-10 mM.

11. The method of claim 3 wherein the B27 Supplement is present at a concentration of about 1×, the bFGF is present at a concentration of about 10 mg/mL, the EGF is present at a concentration of about 20 mg/mL, and the hydrocortisone is present at a concentration of about 1 mM.

12. The method of claim 4 wherein the medium comprises Penicillin at a concentration of about 1,000 Units/mL and Streptomycin at a concentration of about 100 μg/mL.

13. The method of claim 1 wherein the isolated cells are sorted for the presence of at least one marker selected from the group consisting of NESTIN, BMI1, PTEN, and GFAP.

14. A GBM organoid comprising BCSCs, the organoid exhibiting endogenous three-dimensional organ architecture.

15. A brain tissue organoid derived in vitro from primary brain normal tissue, wherein the organoid comprises neural tissue and exhibits endogenous three-dimensional organ architecture.

16. A GBM organoid derived in vitro from primary GBM tissue, wherein the organoid comprises BCSCs and exhibits endogenous three-dimensional organ architecture.

17. A cell culture medium supplemented with B27 Supplement, bFGF, and EGF.

18. A cell culture medium supplemented with B27 Supplement, bFGF, EGF, and hydrocortisone.

19. The cell culture medium of claims 17 and 18 further comprising Penicillin and Streptomycin.

20. A kit comprising the cell culture medium of claim 19.

21. A method for identifying an agent having anticancer activity against GBM cells from a patient comprising selecting at least one test agent, contacting a plurality of GBM organoids derived from GBM cells from the patient with the test agent, determining the number of GBM organoids in the presence of the test agent and the absence of the test agent, and identifying an agent having anticancer activity if the number or growth of the organoids derived from GBM cells from the patient is less in the presence of the agent than in the absence of the agent.

22. A method of personalized treatment for GBM in a subject comprising: selecting at least one form of treatment, contacting a plurality of GBM organoids with the form of treatment, wherein the organoids are derived from GBM cells from the subject, determining the number of GBM organoids in the presence of the treatment and the absence of the treatment, and selecting the treatment if the number or growth of the GBM organoids is less in the presence of the treatment than in the absence of the treatment.

23. The method of claim 22 further comprising treating the subject with the selected treatment.

24. A method of personalized treatment for brain disorders in a subject comprising: selecting normal brain cells to generate organoids, wherein the organoids are derived from normal brain cells from the subject, or HLA-matched donors, generating normal patient-specific or HLA-matched brain organoids, and using such organoids to identify agents that induce neural tissue regeneration and for personalized therapies for brain disorders.

25. A humanized mouse engrafted with components of a patient's immune system and comprising a GBM organoid derived from the patient's GBM cell grafted into the mouse.

26. The method of claim 21, further comprising providing a mouse engrafted with GBM cells from the patient and containing a tumor formed from the GBM cells; administering the identified agent having anticancer activity to the mouse; and determining if the tumor size is reduced in the presence of the identified agent.

27. The method of claim 21, further comprising providing a humanized mouse engrafted with components of a patient's immune system and GBM cells from the patient and containing a tumor formed from the GBM cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered.

28. The method of claim 21 or 27, further comprising providing a humanized mouse engrafted with GBM cells from the patient and containing a tumor formed from the GBM cells; administering a control agent to the humanized mouse engrafted with GBM cells from the patient; and comparing the size of the tumor in the humanized mouse engrafted with GBM cells from the patient to the size of the tumor in the mouse in which the identified agent was administered; and determining if the size of the tumor in the mouse in which the identified agent was administered is reduced relative to the size of the tumor in the humanized mouse engrafted with GBM cells from the patient.

29. The method of any one of claims 21-24 and 26-28, wherein the organoids exhibit endogenous three-dimensional organ architecture.
